(19) United States
(12) Patent Application Publication
(10) Pub. No.: US 2017/0000322 A1
(43) Pub. Date: Jan. 5, 2017

(54) INTRA-ILLUMINATING DEVICE

(71) Applicant: Christopher Fleming, Costa Mesa, CA (US)

(72) Inventor: Christopher Fleming, Costa Mesa, CA (US)

(21) Appl. No.: 14/999,816

(22) Filed: Jul. 1, 2016

Related U.S. Application Data
(60) Provisional application No. 62/187,716, filed on Jul. 1, 2015.

(51) Int. Cl. A61B 1/06; A61B 1/31; A61B 1/00; A61B 1/04; A61B 1/12; A61B 1/32

(52) U.S. Cl. CPC A61B 1/0684; A61B 1/128; A61B 1/32; A61B 1/00029; A61B 1/04; A61B 1/00004; A61B 1/31; A61B 1/00066; A61B 2017/00283

(57) ABSTRACT

An Intra-Illuminating means to provide heat-free light in an improved speculum to improve outcomes of procedures. Likewise disclosed is a computer system including telemedicine linkages.

### CROSS REFERENCE TO RELATED APPLICATIONS

This application claims the benefit of and priority to U.S. Provisional Patent Application No. 62/187,716, filed Jul. 1, 2015, the entire contents of which are incorporated herein by reference, by the inventor and assignee of the both filings.

### FIELD OF THE DISCLOSURE

The present disclosure relates to medical devices for being emplaced into, and illuminating challenging lumens. In particular, the instant disclosure makes novel combinations of cool lighting elements with anoscopes operatively functional according to the instant teachings, to better address long felt needs.

It is known that challenging body lumens often require modified tools to enable and facilitate allopathic and all other manner of surgeries. Prior to the instant teachings, this need remained unaddressed.

### BACKGROUND OF THE DISCLOSURE

Among the listed patents, each of the following was examined and found to be distinct from the instant disclosure.

U.S. Pat. No. 6,428,473 discloses an illuminated rectal retractor, which does not address lighting needs proximal to the surgical site adequately.

U.S. Pat. No. 8,602,971 disclosures an illumination and vision invention for endoscopy, not effective for anoscopy.

U.S. Pat. No. 6,595,917 discloses a speculum with included light, however, in practice, the illumination is not powerful or target enough to work as claimed.

U.S. Pat. No. 7,967,746 discloses a rectoscope having a light-emitting elements, however, they fail to support procedures with a port.

U.S. Pat. No. 6,616,603 discloses an anoscope with light.

U.S. Pat. No. 4,619,248 discloses a light attachment for speculum.

U.S. Pat. No. 8,267,855 discloses an illuminated medical instrument.

U.S. Pat. No. 4,502,468 discloses an adjustable speculum with incorporated lighting system.

United States Design Patent 124,597 illustrates an ornamental design for a surgical speculum.

Foreign Patent Document WO2011081335 discloses a surgical apparatus for transanal surgery.

### OBJECTS AND SUMMARY OF THE DISCLOSURE

Briefly stated, intra-Illuminating means provide heat-free light in an improved speculum to improve outcomes of procedures.

According to embodiments, there is disclosed a system for providing light to lumens during procedures, which comprises, in combination at least an improved housing lights connected to a power source, illuminable by attenuation of power to a series of light emitting diodes, the speculum further comprising guide means for placement, part at the distal end or a procedure facilitating space, for allowing operative interventions, and, an optional power source, for providing heat-free lighting to the area to be illuminated.

According to the embodiment, there is disclosed a process for illuminating the anal cavity, comprising, providing a speculum having a handle and guide means, cool lighting and an operative space, linked to a power source, inserting the speculum in a patient's lumen, attenuating the lighting to provide enough to visualize objects to be managed, performing the required procedure; and removing the speculum; along with repeating as many times as needed.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used.

Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

### BRIEF DESCRIPTION OF THE DRAWINGS

FIG. 1 is a schematic depiction of the instant speculum;

FIG. 2 shows the other aspects of the system; and

FIG. 3 is a flowchart of the present inventions functional steps.

### DETAILED DESCRIPTION OF THE INVENTIONS

The present inventor has discovered how to provide comfort for those having anoscopes and the like devices inserted for procedures into their anal cavities. Those of skill understand the computer interface and data transfer are inherent in the instant teachings and known to be important. Further disclosed is linkage of the same to telemedical systems devices and portals.

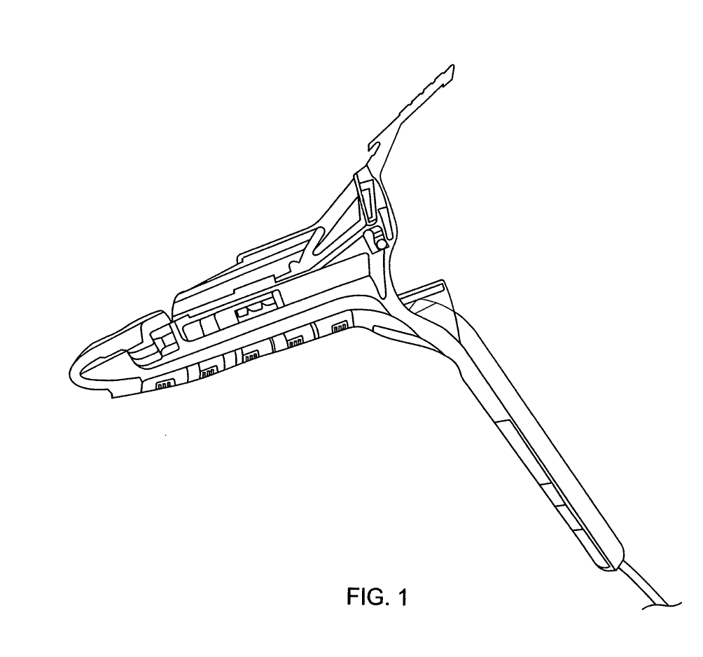

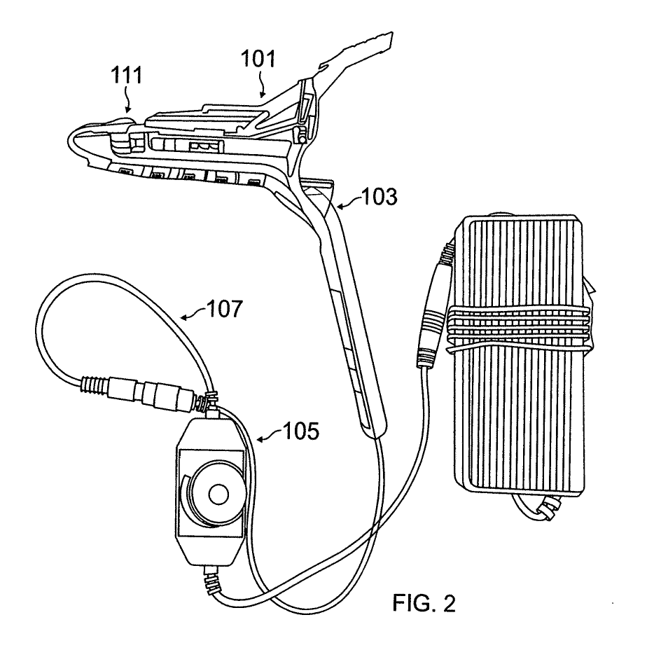

Referring now to FIG. 1 and FIG. 2, the Intra-Illuminating Device of the present invention includes handle/guide-means 101, lighting elements Section 103, and power supply 107, Attenuator 105 allows for lighting to be increased. Part 111 allows for suturing for example.

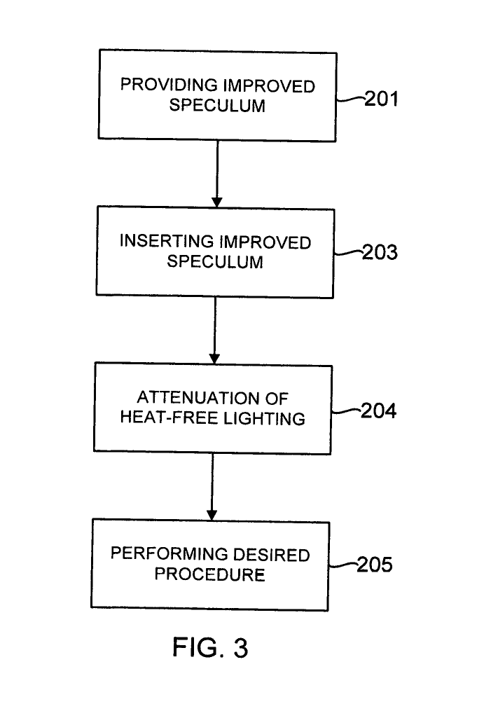

Referring now to FIG. 3, an improved cool-lighted speculum is provided 201, inserted 203, the LED settings adjusted as needed 204, and procedure performed 205. Optionally, the steps are repeated and/or done by a computer.

Part 111, allows for example a surgeon to suture hemorrhoids into the anal canal, which handle/guide-means 101 and lighting elements Section 103 illuminate the way, without generating heat.

Thus, it is respectfully submitted that a computer system for providing light to lumens during procedures, includes at least an adequately resourced and powered processor and includes in combination, an improved medical device defined by, offering for consideration both the software and hardware namely, at least an improved housing having LED (light emitting diode) lights connected to a power source, physically or wirelessly, illuminable by attenuation of power to a the series of light emitting diodes, the speculum further comprising guide means for placement, part of the distal end is a procedure facilitating space, for allowing operative, interventions, and, a power source, for providing heat-free lighting to the area to be illuminated, as set forth, either physically or wirelessly connected to the same.

Those skilled in the art further readily contemplate connection wirelessly or actually to telemedical information ports such as known the those skilled in the art.

All definitions, all defined and used herein, should be understood to control over dictionary definitions, definitions in documents incorporated by reference, and/or ordinary means of the defined terms.

The indefinite articles “a” and “an,” as used herein in the specification and in the claims, unless clearly indicated to the contrary, should be understood to mean “at least one.”

The phase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

Reference throughout this specification to “one embodiment” or “an embodiment” or similar language means that a particular feature, structure, or characteristic described in connection with the embodiment is included in at least one embodiment of the present invention. Thus, appearances of the phrases “in one embodiment” or “in an embodiment” and similar throughout this specification may, but do not necessarily, all refer to the same embodiment.

Furthermore, the described features, structures, or characteristics of the invention may be combined in any suitable manner in one or more embodiments. In the following description, numerous specific details are provided to provide a thorough understanding of embodiments of the invention. One skilled in the relevant art will recognize, however, that the invention may be practiced without one or more of the specific details, or with other methods, components, materials, and so forth. In other instances, well-known structures, materials, and so forth. In other instances, well known structures, materials, or operations are not shown or described in detail to avoid obscuring aspects of the invention.

The schematic flow chart diagrams included herein are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may be or may not strictly adhere to the order of the corresponding steps shown.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

As one skilled in the art would recognize as necessary or best-suited for performance of the methods of the invention, a computer system or machines of the invention include one or more processors (e.g., a central processing unit (CPU) a graphics processing unit (GPU) or both), a main memory and a static memory, which communicate with each other via a bus.

A processor may be provided by one or more processors including, for example, one or more of a single core or multi-core processor (e.g., AMD Phenom II X2, Intel Core Duo, AMD Phenom II X4, Intel Core i5, Intel Core i & Extreme Edition 980X, or Intel Xeon E7-2820).

An I/O mechanism may include a video display unit (e.g., a liquid crystal display (LCD) or a cathode ray tube (CRT)), an alphanumeric input device (e.g., a keyboard), a cursor control device (e.g., a mouse), a disk drive unit, a signal generation device (e.g., a speaker), an accelerometer, a microphone, a cellular radio frequency antenna, and a network interface device (e.g., a network interface card (NIC), Wi-Fi card, cellular modem, data jack, Ethernet port, modem jack, HDMI port, mini-HDMI port, USB port), touchscreen (e.g., CRT, LCD, LED, AMOLED, Super AMOLED), pointing device, trackpad, light (e.g., LED), light/image projection device, or a combination thereof.

Memory according to the invention refers to a non-transitory memory which is provided by one or more tangible devices which preferably include one or more machine-readable medium on which is stored one or more sets of instructions (e.g., software) embodying any one or more of the methodologies or functions described herein. The software may also reside, completely or at least partially, within the main memory, processor, or both during execution thereof by a computer within system, the main memory and the processor also constituting machine-readable media. The software may further be transmitted or received over a network via the network interface device.

While the machine-readable medium can in an exemplary embodiment be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that cause the machine to perform any one or more of the methodologies of the present invention. Memory may be, for example, one or more of a hard disk drive, solid state drive (SSD), an optical disc, flash memory, zip disk, tape drive, “cloud” storage location, or a combination thereof. In certain embodiments, a device of the invention includes a tangible, non-transitory computer readable medium for memory. Exemplary devices for use as memory include semiconductor memory devices, (e.g., EPROM, EEPROM, solid state drive (SSD), and flash memory devices e.g., SD, micro SD, SDXC, SDIO, SDHC cards); magnetic disks, (e.g., internal hard disks or removable disks); and optical disks (e.g., CD and DVD disks).

Furthermore, numerous references have been made to patents and printed publications throughout this specification. Each of the above-cited references and printed publications are individually incorporated herein by reference in their entirety.

In closing, it is to be understood that the embodiments of the invention disclosure herein are illustrative of the principles of the present invention. Other modifications that may be employed are within the scope of the invention. Thus by way of example, but not of limitation, alternative configurations of the present invention may be utilized in accordance with the teachings herein. Accordingly, the present invention is not limited to that precisely as shown and described.

What is claimed is:

1. A system for providing light to lumens during procedures, which comprises, in Combination, an improved medical device defined by:
at least an improved housing having LED (light emitting diode) lights connected to a power source, physically or wirelessly, illuminable by attenuation of power to a the series of light emitting diodes;
the speculum further comprising guide means for placement;
part of the distal end is a procedure facilitating space, for allowing operative interventions; and,
the power source, for providing heat-free lighting to the area to be illuminated, as set forth, either physically or wirelessly connected to the same.

2. The system of claim 1 further comprising at least a processor.

3. The system of claim 2, the at least a processor managing data between at least a user and the operation of the improved medical device.

4. The system of claim 3, further comprising an imaging system.

5. The system of claim 4, further comprising a linked database of patient medical data, and remote access which is HIPAA compliant.

6. A handheld personal digital assistant with an on-board app. For displaying information from the system of claim 5.

7. A kit, comprising, the system of claim 5, and directions for use.

8. The system of claim 1, said LED's comprising a line of white LED's along the lower third of the handle, which is operatively attached to the guide means.

9. The system of claim 8, whereby no excess or tissue damaging heat is delivered to tissue of a patient upon whom the system is being used.

10. A process for illuminating the anal cavity, comprising;
providing a speculum having a handle and guide means, cool lighting and an operative space, linked to a power source;
inserting the speculum in a patient's lumen;
attenuating the lighting to provide enough to visualize objects to be managed;
performing the required procedure; and removing the speculum; along with repeating as many times as needed.

11. The process of claim 10, the cool lighting further comprising at least two light-emitting diodes (LEDs).

12. The process of claim 11, said LED's further comprising a line of white LED's along the lower third of the handle, which is operatively attached to the guide means.

13. The process of claim 12, whereby adequate lighting is provided to address all anal procedures, without heat from the lighting mechanism.

14. An anoscope with a speculum having a handle and guide means further comprising cool-light LED's emplaced within the handle.

15. The anoscope of claim 14, said LED's further comprising a line of white LED's along the lower third of the handle, which is operatively attached to the guide means.

16. The anosocope of claim 15, whereby adequate lighting is provided to address address all anal procedures, without heat from the lighting mechanism.
